(19) United States
(12) Patent Application Publication
(10) Pub. No.: US 2018/0291059 A1
(43) Pub. Date: Oct. 11, 2018

(54) IMMUNOAFFINITY PURIFICATION OF ANTIBODIES USING MIMETOPES

(71) Applicant: Abreos Biosciences, Inc., La Jolla, CA (US)

(72) Inventor: Bradley T. Messmer, San Diego, CA (US)

(21) Appl. No.: 15/944,099

(22) Filed: Apr. 3, 2018

Related U.S. Application Data
(60) Provisional application No. 62/481,282, filed on Apr. 4, 2017.

(51) Int. Cl. C07K 1/22; C07K 16/28

(52) U.S. Cl. CPC C07K 1/22; C07K 2317/24; C07K 16/2842

(57) ABSTRACT

Provided herein are novel methods and matrices for the purification an antibody from a starting material using a mimetope. The method comprises contacting an immunoaffinity chromatography matrix, comprising a solid support and a mimetope immobilized to the solid support, wherein the antibody in the starting material binds to the immobilized mimetope and altering the conditions of the immunoaffinity chromatography matrix to unbind the antibody from the mimetope. The matrix comprises a solid support and a mimetope immobilized to the solid support.

### CROSS-REFERENCE

This application claims priority to U.S. Provisional Application No. 62/481,282, filed Apr. 4, 2017, which is entirely incorporated herein by reference.

### BACKGROUND OF THE INVENTION

Antibodies and other polypeptides are widely used in a variety of applications, from use as therapeutic agents to use as reagents in biological assays. Purification of the complex mixtures in which antibodies are produced to high purity, homogenous antibody preparations has been a bottleneck in the antibody purification process and therefore a target for improvement. Affinity chromatography-based processes have been shown to be the most selective and efficient methods of purification. However, existing affinity chromatography based methods can be harsh, impacting the quality of the resulting purified antibodies. There exists a need for development of milder yet effective means of eluting antibodies during the purification process.

### SUMMARY OF THE INVENTION

Disclosed herein, in certain embodiments, are methods of purifying an antibody from a starting material, comprising: (a) contacting an immunoaffinity chromatography matrix comprising: (i) a solid support; and (ii) a mimetope immobilized to the solid support; wherein the antibody in the starting material binds to the immobilized mimetope; and (b) altering the conditions of the immunoaffinity chromatography matrix to unbind the antibody from the mimetope. In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab. In some embodiments, the starting material is derived from a complex mixture. In some embodiments, the complex mixture comprises plasma, serum, ascites fluid, cell culture medium, egg yolk, plant extracts, bacterial culture, hybridoma culture, or yeast culture. In some embodiments, the plasma is from a human. In some embodiments, the human has developed immunity against a pathogen. In some embodiments, the pathogen is a virus. In some embodiments, the complex mixture comprises a hybridoma culture. In some embodiments, the solid support comprises a natural polymer, a synthetic polymer, an inorganic material, or a combination thereof. In some embodiments, the natural polymer comprises agarose, dextran, cellulose, starch, pectin, mucin, chitin, alginate, gelatin, or a combination thereof. In some embodiments, the synthetic polymer comprises polyamide, polyacrylamide, polymethacrylamide, polyimide, polyesters, polyether, polyvinylalcohol, polyvinylether, polystyrene, polyalkene, polyethylene, polyacralate, polymethacralate, polyethylene, polypropylene, polyfluoroethylene, polyethyleneoxy, polycarbonates, or a combination or co-polymer thereof. In some embodiments, the inorganic material comprises silica, reverse-phase silica, metal silicate, controlled pore glass (CPG), metal oxide, sulfide, or a combination thereof. In some embodiments, the solid support comprises a bead. In some embodiments, the bead is magnetic. In some embodiments, the bead comprises magnetic nanoparticles. In some embodiments, the magnetic nanoparticles comprise iron oxide (FeO). In some embodiments, the bead has a particle size of about 1 μm to 50 μm. In some embodiments, the bead has a particle size of about 10 μm. In some embodiments, the mimetope has a length of 7 to 23 amino acids. In some embodiments, the mimetope comprises a disulfide bridge. In some embodiments, the mimetope has low affinity to the antibody. In some embodiments, the mimetope further comprises a linker sequence. In some embodiments, the linker sequence is 3 or 4 amino acids in length. In some embodiments, the linker sequence is polylysine, polysaspartic acid, polyglutamic acid, or polyarginine. In some embodiments, the method further comprises identifying the mimetope using a phage-displayed peptide library. In some embodiments, immobilizing the mimetope to the solid support comprises synthesizing the mimetope directly on the solid support. In some embodiments, solid-phase peptide synthesis is used to synthesize the mimetope directly on the solid support. In some embodiments, immobilizing the mimetope to solid support comprises covalently binding the mimetope to the solid support. In some embodiments, contacting the matrix with the starting material comprises administering to the matrix a binding buffer to optimize binding of the antibody to the mimetope. In some embodiments, contacting the matrix with the starting material comprises administering to the matrix a wash buffer to remove components of the starting material that have not bound to the mimetope. In some embodiments, altering the conditions of the matrix to unbind the antibody comprises changing a solvent condition. In some embodiments, the solvent condition is pH, ionic strength, or polarity. In some embodiments, altering the conditions of the matrix to unbind the antibody comprises administering to the matrix a reducing agent. In some embodiments, the reducing agent is β-mercaptoethanol, dithiothreitol, or Tris (2-Carboxyethyl) phosphine hydrochloride. In some embodiments, altering the conditions of the matrix to unbind the antibody comprises administering to the matrix a displacer agent which binds to the antibody. In some embodiments, the displacer agent is an unbound mimetope. In some embodiments, the unbound mimetope is identical to the immobilized mimetope. In some embodiments, the unbound mimetope is not identical to the immobilized mimetope. In some embodiments, the unbound mimetope has a higher affinity to the antibody relative to the immobilized mimetope.

Disclosed herein, in certain embodiments, are matrices for immunoaffinity purification of an antibody, comprising: a. a solid support; and b. a mimetope immobilized to the solid support. In some embodiments, the solid support comprises a natural polymer, a synthetic polymer, an inorganic material, or a combination thereof. In some embodiments, the natural polymer comprises agarose, dextran, cellulose, starch, pectin, mucin, chitin, alginate, gelatin, or a combination thereof. In some embodiments, the synthetic polymer comprises polyamide, polyacrylamide, polymethacrylamide, polyimide, polyesters, polyether, polyvinylalcohol, polyvinyl ether, polystyrene, polyalkene, polyethylene, polyacralate, polymethacralate, polyethylene, polypropylene, polyfluoroethylene, polyethyleneoxy, polycarbonates, or a combination or co-polymer thereof. In some embodiments, the inorganic material comprises silica, reverse-phase silica, metal silicate, controlled pore glass (CPG), metal oxide, sulfide, or a combination thereof. In some embodiments, the solid support comprises a bead. In some embodiments, the bead is magnetic. In some embodiments, the bead comprises magnetic nanoparticles. In some embodiments, the magnetic nanoparticles comprise iron oxide (FeO). In some embodiments, the bead has a particle size of about 1 μm to 50 μm. In some embodiments, the bead has a particle size of about 10 μm. In some embodiments, the mimetope has a length of 7 to 23 amino acids. In some embodiments, the mimetope comprises a disulfide bridge. In some embodiments, the mimetope has low affinity to the antibody. In some embodiments, the mimetope further comprises a linker sequence. In some embodiments, the linker sequence is 3 or 4 amino acids in length. In some embodiments, the linker sequence is polylysine, polysaspartic acid, polyglutamic acid, or polyarginine.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

The bottleneck in commercial production of antibodies occurs at the antibody purification step. Additionally, downstream processing accounts for 50-80% of the total manufacturing costs of therapeutic antibodies. Mild but effective elution techniques have the ability to decrease costs and time associated with purification, as well as improve the quality of the resulting product.

Provided herein, are matrices and methods for the immunoaffinity purification of an antibody using a mimetope. The methods described herein include methods for purifying an antibody from a starting material comprising the steps of obtaining an immunoaffinity chromatography matrix comprising a solid support and a mimetope immobilized to the solid support, contacting the immunoaffinity chromatography matrix with a starting material containing an antibody of interest, allowing binding to occur between the mimetope immobilized to the solid support and the antibody of interest, and altering the conditions of the immunoaffinity chromatography matrix to unbind the antibody from the mimetope immobilized to the solid support.

### Certain Definitions

Unless otherwise defined, all technical terms used herein have the same meaning as commonly understood by one of ordinary skill in the art. As used in this specification and the appended claims, the singular forms “a,” “an,” and “the” include plural references unless the context clearly dictates otherwise. Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value.

As used herein, a “mimetope” is a determinant which is recognized by the same binding molecule, such as an antibody, as a particular “epitope” but which has a different composition from the “epitope.” For example, a binding molecule can be an antibody which recognizes (i.e., binds to) an epitope comprising a linear sequence of amino acids. A “mimetope” of this epitope comprises a different linear sequence of amino acids but which is still recognized by the same antibody. In some embodiments, the mimetope is a Veritope™.

As used herein, “polypeptide” and “peptide” are used broadly to refer to macromolecules comprising linear polymers of natural or synthetic amino acids. Polypeptides may be derived naturally or synthetically by standard methods known in the art. While the term “polypeptide” and “peptide” are synonymous, the term “polypeptide” generally refers to molecules of greater than 40 amino acids, while the term “peptide” generally refers to molecules of 2 to 40 amino acids. In some embodiments, the peptide is a mimetope.

The term “antibody” as used herein refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and portions thereof; including, for example, an immunoglobulin molecule, a polyclonal antibody, a monoclonal antibody (mAb), a recombinant antibody, a chimeric antibody, a humanized antibody, a CDR-grafted antibody, F(ab)₂, Fv, scFv, IgGΔCH₂, F(ab′)2, scFv2CH₃, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM), a modified antibody, and a synthetic antibody (including, without limitation non-depleting IgG antibodies, T-bodies, or other Fc or Fab variants of antibodies).

As used herein, the terms “sample” and “biological sample” refer to any sample suitable for the methods provided by the present invention. In one embodiment, the biological sample of the present invention is a physiological fluid, for example, whole blood or fraction thereof (e.g., serum or plasma), urine, spinal fluid, saliva, ejaculate, and stool.

As used herein, the term “solid support” refers to any solid phase material upon which a polypeptide, such as a mimetope, is synthesized or attached, such as conjugation via covalent bond. Solid support encompasses terms such as “resin,” “solid phase,” and “support.”

### Affinity Chromatography Matrix

Disclosed herein, in certain embodiments, are matrices for immunoaffinity purification of an antibody, comprising: (a) a solid support; and (b) a mimetope immobilized to the solid support. Mimetopes are described in U.S. Pat. No. 9,250,233, which is hereby incorporated by reference for purposes of describing mimetope production methods.

### Solid Support

In some embodiments, the configuration of the solid support is in the form of beads, spheres, particles, granules, or a surface. In some embodiments, the surface is planar, substantially planar, or non-planar. In some embodiments, the solid support is porous or non-porous. In some embodiments, the solid support has swelling or non-swelling characteristics. In some embodiments, the solid support is configured in the form of a well, depression, or other vessel.

In some embodiments, the solid support comprises a natural polysaccharide, a synthetic polymer, an inorganic material, or a combination thereof. In some embodiments, the solid support comprises a natural polymer. In some embodiments, the natural polymer comprises agarose, cellulose, cellulose ethers (e.g. hydroxypropyl cellulose, carboxymethyl cellulose), starches, gums (e.g. guar gum, gum arabic, gum ghatti, gum tragacanth, locust bean gum, xanthan gum), pectin, mucin, dextran, chitin, chitosan, alginate, carrageenan, heparin, gelatin, or a combination thereof. In some embodiments, the solid support comprises a synthetic polymer. In some embodiments, the synthetic polymer comprises a polymer selected from polyamide (e.g. polyacrylamide, polymethacrylamide), polyimide, polyesters, polyether, polymeric vinyl compounds (e.g. polyvinylalcohol, polyvinylether, polystyrene), polyalkene, polyethylene, polyacralate, polymethacralate, polyethylene, polypropylene, polyfluoroethylene, polyethyleneoxy, polycarbonates, and a combination or co-polymer thereof. In some embodiments, the natural polymer or the synthetic polymer is cross-linked. In some embodiments, the solid support comprises an inorganic material. In some embodiments, the inorganic material comprises silicious materials (e.g. silicon dioxide including amorphous silica and quartz), silica, reverse-phase silica, metal silicate, controlled pore glass (CPG), metal oxide (e.g. TiO₂), sulfide, or a combination thereof.

In some embodiments, the solid support is a commercially available solid support. In some embodiments, the commercially available solid support is Affi-Gel (BioRad), Affinica Agarose/Polymeric Supports (Schleicher and Schuell), AvidGel (BioProbe), Bio-Gel (BioRad), Fractogel (EM Separations), HEMA-AFC (Alltech), Reacti-Gel (Pierce), Sephacryl (Pharmacia), Sepharose (Pharmacia), Superose (Pharmacia), TentaGel (Rapp Poplymere), Trisacryl (IBF), TSK Gel Toyopearl (TosoHaas), Ultagel (IBF), Avid Gel CPG (BioProbe), HiPAC (ChromatoChem), Protein-Pak Affinity Packing (Waters), Ultraaffinity-EP (Bodman), Emphaze (3M Corp./Pierce), or POROS (ABI/PerSeptive Biosystems).

In some embodiments, the solid support comprises pores. In some embodiments, the pore has a pore diameter of about 300 to about 500 Å. In some embodiments, the pore diameter is about three to about five times the diameter of the mimetope to be immobilized to the solid support.

In some embodiments, the solid support is a bead. In some embodiments, the bead is manufactured from any suitable material. In some embodiments, the bead is made of a resin that is a graft copolymer of a crosslinked polystyrene matrix and polyethylene glycol (PEG), such as TentaGel™ beads (Rapp Polymere GmbH). Without being bound to any theory, PEG, a main constituent of the bead material, is often used to limit non-specific adsorption of proteins to surfaces and particles.

In some embodiments, the beads are 1 μm to 1000 μm in diameter. In some embodiments, the bead diameter is about 1, 2, 3, 4, 5, 6, 7, 8, 9, 10, 15, 20, 25, 30, 35, 40, 45, 50, 60, 70, 80, 90, 100, 200, 300, 400, 500, 750, or 1000 μm. In some embodiments, the bead diameter is about 10 μm. In some embodiments, beads of two or more diameters are used.

In some embodiments, the bead comprises a nanoparticle. As used herein, the term “nanoparticle” refers to any particle having a diameter of less than 1000 nanometers (nm). In some embodiments, the nanoparticle is optically or magnetically detectable. In some embodiments, the nanoparticle has a diameter of 200 nm or less. In some embodiments, the nanoparticle has a diameter of about 100, 50, 40, or 30 nm or less. In some embodiments, the nanoparticle has a diameter of about 5 to about 25 nm. In some embodiments, the nanoparticle is a quantum dot, such as bright, fluorescent nanocrystals with physical dimensions small enough such that the effect of quantum confinement gives rise to unique optical and electronic properties. In some embodiments, the nanoparticle is a metal nanoparticle.

In some embodiments, the nanoparticle is magnetic. For example, magnetization of the beads allows for one to use automated handling technologies to wash and manipulate the beads during the detection process. Additionally when dealing with fewer beads, it is easier to recover a greater number of beads for measurement when the beads are magnetized. Because one can use a lower number of beads, the signal per bead is higher, thus improving the signal response and thus increasing sensitivity.

As used herein, magnetic nanoparticles refer to magnetically responsive particles that contain one or more metals or oxides or hydroxides thereof. Magnetically responsive materials of interest include paramagnetic materials, ferromagnetic materials, ferrimagnetic materials, and metamagnetic materials. In some embodiments, any magnetic nanoparticle are used, so long as the particles are dispersed or suspended in an aqueous medium and have the ability to be separated from a dispersion liquid or a suspension through application of a magnetic field. In some embodiments, magnetic nanoparticles include, for example, a salt, oxide, boride or sulfide of iron, cobalt or nickel; and rare earth elements having high magnetic susceptibility (e.g., hematite and ferrite). Specific examples of magnetic nanoparticles include iron, nickel, and cobalt, as well as metal oxides such as Fe₃O₄, BaFe₁₂O₁₉, CoO, NiO, Mn₂O₃, Cr₂O₃, and CoMnP. Additional examples of iron oxides particularly include a magnetite, a maghemite, and a mixture thereof.

In some embodiments, the solid support is a magnetic bead. In some embodiments, wherein the solid support is a magnetic bead, the quantity of magnetically responsive material in the bead is not critical and varies over a wide range. In some embodiments, this quantity affects the density of the bead, however, both the quantity of magnetically responsive material and the particle size affects the ease of maintaining the bead in suspension for purposes of achieving maximal contact between the liquid and solid phase and for facilitating flow cytometry. In some embodiments, an excessive quantity of magnetically responsive material in the bead produces autofluorescence at a level high enough to interfere with the assay results. It is therefore preferred that the concentration of magnetically responsive material be low enough to minimize any autofluorescence emanating from the material. In some embodiments, the magnetically responsive material in a bead ranges from about 1% to about 75% by weight of the particle as a whole. In some embodiments, the weight percent range is from about 2% to about 50%. In some embodiments, the weight percent range is from about 3% to about 25%. In some embodiments, the weight percent range is from about 5% to about 15%. In some embodiments, the magnetically responsive material is dispersed throughout the polymer, applied as a coating on the polymer surface or as one of two or more coatings on the surface, or incorporated or affixed in any other manner that secures the material in the polymer matrix.

### Mimetope

In some embodiments, a mimetope is immobilized to the solid support. In some embodiments, immobilizing the mimetope to the solid support does not affect the activity of the binding site of the mimetope or the accessibility of the binding site to the antibody.

In some embodiments, phage-displayed peptide libraries are used to select peptide sequences that mimic the target antigen of a given mAb. In some embodiments, peptide libraries displayed on bacteriophage are routinely used to identify peptide epitopes, or mimetopes (also referred to as VERITOPES™), recognized by antibodies. In some embodiments, the mimetopes identified using the phage-displayed peptide libraries are specific and compete with the antigen for antibody binding.

In some embodiments, the mimetope has a length of about 2 to about 40 amino acids. In some embodiments, the mimetope has a length of about 5 to about 26 amino acids. In some embodiments, the mimetope has a length of about 7 to about 23 amino acids. In some embodiments, the mimetope has a length of about 10 to about 23 amino acids. In some embodiments, the mimetope has a length of about 12 to about 23 amino acids. In some embodiments, the mimetope has a length of 7 amino acids. In some embodiments, the mimetope has a length of 10 amino acids. In some embodiments, the mimetope has a length of 12 amino acids. In some embodiments, the mimetope has a length of 23 amino acids.

In some embodiments, the sequence of the mimetope of interest is identified by sequencing the relevant portion; e.g., the binding site identified in the panned phage genomes.

In some embodiments, the mimetope is directly synthesized on the solid support. In some embodiments, the mimetope is directly synthesized on the solid support by solid-phase peptide synthesis (SPPS). In general, two strategies for the synthesis of peptide chains by SPSS are known in the art; stepwise solid-phase peptide synthesis, and solid-phase fragment condensation. In stepwise SPPS, the C-terminal amino acid is in the form of an N-α-protected side-chain, and the protected reactive derivative is covalently coupled either directly or by means of a suitable linker to a solid support, which is typically swollen in an organic solvent. In some embodiments, the N-α-protected group is removed, and the subsequent protected amino acids are added in a stepwise fashion.

When the desired mimetope chain length has been obtained, the side-chain protective groups are removed. In some embodiments, the mimetope is cleaved from the solid support. In some embodiments, the mimetope is not cleaved from the solid support. In some embodiments, the solid support from which the mimetope has not been cleaved is used as the solid support for the methods described herein. In some embodiments, removal of the protective group and cleavage is done in separate steps or at the same time.

In solid-phase fragment condensation, the target sequence is assembled by consecutive condensation of fragments on a solid support using protected fragments prepared by stepwise SPPS. Additional conventional methods of performing SPSS include split and mix synthesis, reagent mixture synthesis, and in situ parallel synthesis.

Typically, two coupling strategies are used to perform SPPS, tert-butyloxycarbonyl (Boc) and fluorenylmethyloxycarbonyl (Fmoc), which are based on the use of different N-α-protective groups and matching side-chain protective groups. The Boc approach utilizes Boc as the N-α-protective group, versus Fmoc. While the Boc and Fmoc strategies have been used for essentially all current practical peptide synthesis, other N-α-protective groups have been proposed.

In some embodiments, the mimetope is attached to the solid support using electrostatic attraction, specific affinity interaction, hydrophobic interaction, or covalent bonding.

In some embodiments, the mimetope is covalently attached to the solid support. In some embodiments, a functional group for attachment to the mimetope is incorporated into the polymer structure of the solid support by conventional means, including the use of monomers that contain the functional groups, either as the sole monomer or as a co-monomer. Examples of suitable functional groups are amine groups (—NH₂), ammonium groups (—NH₃₊ or —NR₃₊), hydroxyl groups (—OH), carboxylic acid groups (—COOH), carbonyl groups (—C═O), sulfhydryl groups (—SH), and isocyanate groups (—NCO). Useful monomers for introducing carboxylic acid groups into polyolefins, for example, are acrylic acid and methacrylic acid. In some embodiments, the solid support is activated with a compound that is reactive toward one or more functional groups. In some embodiments, the compound reactive toward one or more functional groups is cyanogen bromide (CNBr), carbonyl diimidazole (CDI), carbodiimide, epoxy, divinyl sulfone, toluene sulfonyl chloride, or N-hydroxysuccinimide ester (NHS).

In some embodiments, the mimetope further comprises a linker, or spacer, peptide. In some embodiments, the linker is about 1, 2, 3, 4, 5, 6, 7, 8, 9, or 10 amino acids in length. In some embodiments, the linker is 3 amino acids in length. In some embodiments, the linker is 4 amino acids in length. In some embodiments, the linker facilitates attachment or synthesis. In some embodiments, the linker is used as a means of increasing the density of reactive groups on the solid phase surface. In some embodiments, the linker is used to decrease steric hindrance. Examples of suitable linkers are polylysine, polyaspartic acid, polyglutamic acid, and polyarginine.

In some embodiments, the mimetope comprises a nucleic acid modification. In some embodiments, the nucleic acid modification is a cysteine modification, a lysine modification, or a combination thereof. In some embodiments, the cysteine modification is accomplished via mixed disulfide formation, alkylation with α-halo carbonyl compounds, or addition of maleimide groups. In some embodiments, the lysine modification is an N-terminal α-amino group modification or an ε-amino group modification. In some embodiments, the lysine modification is accomplished by an NETS-ester or an isothiocyanate.

### Methods of Purifying an Antibody

Disclosed herein, are methods of purifying an antibody from a starting material, comprising: (a) contacting an immunoaffinity chromatography matrix comprising (i) a solid support; and (ii) a mimetope immobilized to the solid support, with the starting material such that the antibody binds to the immobilized mimetope; and (b) altering the conditions of the immunoaffinity chromatography matrix to unbind the antibody from the mimetope.

### Antibodies

In some embodiments, the antibody is a monoclonal antibody, a recombinant antibody, a chimeric antibody, or a humanized antibody. In some embodiments, the antibody is a monoclonal antibody (mAb). In some embodiments, the antibody is a mouse antibody or a human antibody.

Examples of monoclonal antibodies include, but are not limited to 3F8, Abagovomab, Abatacept, Abciximab, ACZ885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab, Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab, Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

### Starting Material Preparation

In some embodiments, the method comprises preparing a starting material for administration to the matrix. In some embodiments, the starting material is derived from a complex mixture. In some embodiments, the complex mixture comprises cells expressing the antibody extracellularly or intracellularly. In some embodiments, the preparing the staring material comprises releasing the antibodies from the cells of the complex mixture, administering a denaturing agent to the complex mixture, clarifying the complex mixture to remove contaminants, or a combination thereof.

In some embodiments, the complex mixture is plasma, serum, ascites fluid, cell culture medium, egg yolk, plant extracts, bacterial culture, hybridoma culture, or yeast culture. In some embodiments, the plasma is from a human. In some embodiments, the human has developed immunity against a pathogen. In some embodiments, the pathogen is a virus, a bacterium, a fungus, or a protozoan. In some embodiments, the complex mixture is a hybridoma culture. In some embodiments, the hybridoma culture is derived from a single hybridoma cell. In some embodiments, the hybridoma culture produces a monoclonal antibody (mAb).

In some embodiments, the complex mixture is initially characterized to verify if the antibodies are expressed extracellularly or intracellularly to determine the type of extraction or clarification procedure to use.

In some embodiments, the antibodies are released from the cells of the complex mixture to produce a starting material. In some embodiments, releasing the antibodies comprises disrupting the lipid membrane of the cells of the complex mixture. In some embodiments, disrupting the cell membranes comprises administering osmotic shock, liquid shear pressure (e.g. French press), ultrasonication, homogenization, glass bead milling, repeated freezing and thawing, enzymatic lysis, or a combination thereof. In some embodiments, disrupting the cells yields a suspension of lipid membranes comprising membrane proteins, wherein the membrane proteins comprise the antibody. In some embodiments, the membrane protein is extracted from the lipid membrane to an aqueous environment with the use of a detergent. In some embodiments, detergent is a Brij™35, C12E8, CHAPS, Cymal 7, decyl maltoside, digitonin, dodecyl maltoside (DDM), FOS-choline 12, Hecameg, lauryldimethylamine oxide (LDAO), nonidet P40, nonyl glucoside, octyl glucoside, Tween™ 20, or Triton™ X-100.

In some embodiments, a denaturing agent is administered to the complex mixture. In some embodiments, the denaturing agent is administered to the complex mixture if the antibodies are expressed as aggregates. In some embodiments, the denaturing agent is urea, guanidine hydrochloride, Triton™ X-100, sarcosyl, N-octyl glucoside, or sodium dodecyl sulphate (SDS).

In some embodiments, the complex mixture is clarified to produce a starting material. In some embodiments, clarification of the complex mixture comprises removing contaminants. In some embodiments, clarification of the complex mixture comprises centrifugation, filtration, or precipitation. In some embodiments, precipitation comprises the use of caprylic acid, ammonium sulfate, dextran sulphate, polyvinylpyrrolidine, polyethylene glycol, acetone, polyethyleneimine, protamine sulphate, streptomycin sulphate, or a combination thereof. In some embodiments, precipitation comprises precipitation of the antibodies. In some embodiments, precipitation comprises precipitation of other proteins (e.g. the contaminants).

In some embodiments, the affinity of the mimetope:antibody complex is determined before applying the starting material to the matrix. Affinity referred to is a measure of the strength of interaction between the mimetope and an antibody's antigen binding site. Affinity is measured by the equilibrium dissociation constant (K_{D}). Lower values of K_{D} indicate a higher affinity, and vice versa. In some embodiments, the antibody has affinity for the mimetope of less than about 1.0×10⁻⁵ M. In some embodiments, the dissociation constant is between about 1.0×10⁻⁵ and about 1.0×10⁻⁶ M. In some embodiments, the dissociation constant is between about 1.0×10⁻⁶ and about 1.0×10⁻⁷ M. In other embodiments, the dissociation constant is between about 1.0×10⁻⁷ and about 1.0×10⁻⁸ M. In still other embodiments, the dissociation constant is between about 1.0×10⁻⁸ and about 1.0×10⁻⁹ M. In yet other embodiments, the dissociation constant is more than about 9.9×10⁻¹⁰ M. In some embodiments, affinity is measured using art-known techniques, such as ELISA or BIACORE.

In some embodiments, a mimetope with low affinity to the antibody is preferred. In some embodiments, the use of a low affinity mimetope, in combination with a displacer agent during the elution step, allows the displacer agent to effectively compete with the immobilized mimetope for the antibody. In some embodiments, the displacer agent is an unbound mimetope. In some embodiments, the unbound mimetope is identical to the immobilized mimetope. In some embodiments, the unbound mimetope is not identical to the immobilized mimetope. In some embodiments, the unbound mimetope has a different affinity to the antibody relative to the immobilized mimetope. In some embodiments, the unbound mimetope has a higher affinity to the antibody relative to the immobilized mimetope. In some embodiments, the unbound mimetope has a lower affinity to the antibody relative to the immobilized mimetope.

### Binding/Washing

In some embodiments, the method comprises contacting the starting material with an affinity chromatography matrix comprising a solid support and a mimetope immobilized on the solid support. In some embodiments, contacting the starting material with the matrix results in the antibody in the starting material binding to the immobilized mimetope of the solid support. In some embodiments, contacting the matrix with the starting material comprises administering a binding buffer to the matrix. In some embodiments, contacting the matrix with the starting material comprises administering a wash buffer to the matrix.

In some embodiments, binding of the antibody to the immobilized mimetope is carried out under physiological conditions.

In some embodiments, any suitable binding buffer is used. In some embodiments, the binding buffer provides optimum conditions for binding of the antibody to the mimetope. In some embodiments, the binding buffer has a pH for 7.0 to 7.4. In some embodiments, the binding buffer comprises phosphate buffered saline (PBS), Tris buffered saline (TBS), Tween20, BSA, or a combination thereof. In some embodiments the binding buffer comprises TBS, 0.05% Tween20, and 2.5% BSA. In some embodiments, nonspecific binding interactions are minimized by adjusting the salt concentration of the binding buffer or adding low levels of a detergent to the binding buffer.

In some embodiments, the wash buffer removes components of the starting material that have not bound to the mimetope. In some embodiments, the components comprise protein, lipids, nucleic acids, or other impurities. In some embodiments, the wash buffer does not disturb the binding of the antibody to the mimetope. In some embodiments, the pH or salt concentration (ionic strength) of the wash buffer is adjusted.

In some embodiments, the wash buffer comprises a salt. In some embodiments, the salt is NaCl or MgCl₂. In some embodiments, the wash buffer comprises a detergent. In some embodiments, the detergent is Tween™ 20 or Triton™ X-100. In some embodiments, the wash buffer comprises a blocking agent. In some embodiments, the blocking agent is bovine serum albumin or a mimicking agent. In some embodiments, any suitable wash buffer is used.

In some embodiments, the binding buffer and the wash buffer are identical.

In some embodiments, the method comprises washing the matrix with a second, third, or fourth wash buffer.

In some embodiments, the flow rate of administration of the binding buffer or the wash buffer is paused and then resumed. In some embodiments, the flow rate is paused for about 10 minutes to about 2 hours

### Elution

In some embodiments, the method comprises altering the conditions of the immunoaffinity chromatography matrix to unbind, or dissociate, the antibody from the mimetope. In some embodiments, altering the conditions of the matrix to unbind the antibody comprises administering an elution buffer to the matrix. In some embodiments, unbinding, or dissociating, the mimetope from the antibody comprises altering the pH, altering the ionic strength, denaturing the mimetope and/or the antibody, removal of a binding factor, or competition with a displacer agent.

In some embodiments, the elution buffer dissociates the mimetope from the antibody by altering the pH. In some embodiments, the elution buffer to alter the pH comprises: glycine HCl, citric acid, trimethylamine or triethanolamine, ammonium hydroxide, or glycine NaOH. In some embodiments, the elution buffer dissociates the mimetope from the antibody by altering ionic strength. In some embodiments, the elution buffer to alter the ionic strength comprises: magnesium chloride, lithium chloride, magnesium chloride, potassium chloride, sodium iodide, potassium iodide, sodium thiocyanate, or Tris-acetate. In some embodiments, the elution buffer dissociates the mimetope from the antibody by the use of a detergent or a chaotropic agent that denatures the mimetope and/or antibody. In some embodiments, the elution buffer to denature the mimetope and/or the antibody comprises: guanidine HCl, urea, deoxycholate, ammonium thiocyanate, trifluoroacetate, perchlorate, iodide, chloride, sodium deoxycholate, sarcosyl, or sodium dodecyl sulphate (SDS). In some embodiments, the elution buffer dissociates the mimetope from the antibody by altering polarity. In some embodiments, the elution buffer to alter polarity comprises dioxane or ethylene glycol. In some embodiments, the elution buffer dissociates the mimetope from the antibody by competition with a displacer agent. In some embodiments, the displacer agent is an unbound mimetope. In some embodiments, the unbound mimetope is identical to the immobilized mimetope. In some embodiments, the unbound mimetope is not identical to the immobilized mimetope. In some embodiments, the elution buffer is any suitable elution buffer.

In some embodiments, the mimetope comprises a disulfide bond. In some embodiments, when the mimetope comprises a disulfide bond, the elution buffer comprises a reducing agent. In some embodiments, the reducing agent is β-mercaptoethanol (BME), dithiothreitol (DTT), or Tris (2-Carboxyethyl) phosphine hydrochloride (TCEP HCl).

In some embodiments, the flow rate of administration of the elution buffer is paused and then resumed. In some embodiments, the flow rate is paused for about 10 minutes to about 2 hours.

In some embodiments, the eluted antibody is collected into a neutralization buffer. In some embodiments, the neutralization buffer is Tris-HCl. In some embodiments, the eluted antibody is immediately stored. In some embodiments, the eluted antibody is ultra-filtered, freeze-dried, or precipitated.

In some embodiments, the matrix is optionally cleaned (i.e. regenerated) after elution of the antibody. In some embodiments, cleaning the matrix comprises washing the matrix with solutions able to clean the matrix and/or kill microorganisms. Examples of a solution to wash the matrix include, but are not limited to, 0.1-1.0 M sodium hydroxide; solutions of peracids or hydrogen peroxide; denaturants such as guanidinium hydrochloride; solutions comprising active chlorine such as hypochlorite solutions; organic solvents such as ethanol; detergents; etc. In some embodiments, the matrix is further reused for subsequent antibody purification.

### EXAMPLE

### Example 1: Immunoaffinity Based Purification of Natalizumab Using a Natalizumab-Specific Mimetope

### Identification of a Natalizumab-Specific Mimetope

Mimetope peptides are selected from phage display libraries, some of which contain cysteines flanking the peptide mimetope sequence to increase stability of the peptide through disulfide bond formation. After three rounds of selection with multiple phage display libraries, individual phage plaques are isolated and sequenced. All clones are individually amplified, purified, and their ability to specifically bind natalizumab-coated wells are assessed, and their affinity is measured. The phage clone demonstrating specific, but low affinity binding to natalixumab is chemically synthesized with an N-terminal acetyl modification and a disulfide bridge between cysteines 2 and 10 or cysteines 8 and 16 by a contract peptide manufacturer. The synthetic mimetope is supplied as TFA salt at >84% purity confirmed by mass spec and HPLC.

### Purification of Natalizumab

A hybridoma culture is created from a single hybridoma colony verified to produce natalizumab. Hybridoma culture supernatant is applied to an immunoaffinity chromatography matrix comprising 10 μm TentaGel™ beads upon which the mimetope previously identified with low affinity to natalizumab is directly synthetized using solid phase peptide synthetsis (SPPS). A Tris buffered saline (TBS)+0.05% Tween20+2.5% BSA buffer is administered to the matrix to wash away contaminants and assist with binding efficiency. Given the low affinity of the mimetope to the matrix, a second, unbound mimetope with higher affinity than the immobilized mimetope is administered to the matrix, dissociating the antibody from the immobilized mimetope, and eluting the antibody from the matrix without the use of harsh eluting techniques, avoiding damage to the antibody. The resulting purified antibody composition is immediately ultrafiltered and frozen for storage.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of purifying an antibody from a starting material, comprising the steps of:
a. contacting an immunoaffinity chromatography matrix comprising:
i. a solid support; and
ii. a mimetope immobilized to the solid support;
with the starting material such that the antibody in the starting material binds to the mimetope immobilized to the solid support; and
b. altering the conditions of the immunoaffinity chromatography matrix to unbind the antibody from the mimetope immobilized to the solid support.

2.-12. (canceled)

13. The method of claim 1, wherein the solid support comprises a bead.

14. The method of claim 13, wherein the bead is magnetic.

15. (canceled)

16. (canceled)

17. The method of claim 13, wherein the bead has a particle size of about 1 μm to 50 μm.

18. The method of claim 13, wherein the bead has a particle size of about 10 μm.

19. The method of claim 1, wherein the mimetope immobilized to the solid support has a length of 7 to 23 amino acids.

20. The method of claim 1, wherein the mimetope immobilized to the solid support comprises a disulfide bridge.

21. The method of claim 1, wherein the mimetope immobilized to the solid support has low affinity to the antibody.

22. The method of claim 1, wherein the mimetope immobilized to the solid support further comprises a linker sequence.

23. The method of claim 22, wherein the linker sequence is 3 or 4 amino acids in length.

24. The method of claim 22, wherein the linker sequence is polylysine, polysaspartic acid, polyglutamic acid, or polyarginine.

25. The method of claim 1, wherein the sequence of the mimetope immobilized to the solid support has been identified using a phage-displayed peptide library.

26.-28. (canceled)

29. The method of claim 1, wherein the step of contacting the immunoaffinity chromatography matrix with the starting material comprises administering to the immunoaffinity chromatography matrix a binding buffer to optimize binding of the antibody to the mimetope immobilized to the solid support.

30. The method of claim 1, wherein the step of contacting the immunoaffinity chromatography matrix with the starting material comprises administering to the immunoaffinity chromatography matrix a wash buffer to remove components of the starting material that have not bound to the mimetope immobilized to the solid support.

31. The method of claim 1, wherein the step of altering the conditions of the immunoaffinity chromatography matrix to unbind the antibody from the mimetope immobilized to the solid support comprises changing a solvent condition.

32. The method of claim 31, wherein the solvent condition is pH, ionic strength, or polarity.

33. The method of claim 1, wherein the step of altering the conditions of the immunoaffinity chromatography matrix to unbind the antibody from the mimetope immobilized to the solid support comprises administering to the matrix a reducing agent.

34. The method of claim 33, wherein the reducing agent is (3-mercaptoethanol, dithiothreitol, or Tris (2-Carboxyethyl) phosphine hydrochloride.

35. The method of claim 1, wherein the step of altering the conditions of the immunoaffinity chromatography matrix to unbind the antibody from the mimetope immobilized to the solid support comprises administering to the matrix a displacer agent which binds to the antibody.

36. The method of claim 35, wherein the displacer agent is an unbound mimetope having a higher affinity to the antibody relative to the mimetope immobilized to the solid support.

37.-56. (canceled)
